(19) United States
(12) Patent Application Publication
(10) Pub. No.: US 2024/0293267 A1
(43) Pub. Date: Sep. 5, 2024

(54) TROUSER AND UNDERPANTS SYSTEM WITH AN ABSORBENT FLAP

(71) Applicant: Kim M. Palmen, Stillwater, MN (US)

(72) Inventor: Kim M. Palmen, Stillwater, MN (US)

(21) Appl. No.: 18/588,763

(22) Filed: Feb. 27, 2024

Related U.S. Application Data
(60) Provisional application No. 63/488,404, filed on Mar. 3, 2023.

(51) Int. Cl. A61F 13/496; A61F 13/505; A61F 13/62; A61F 13/70; A61F 13/78

(52) U.S. Cl. CPC A61F 13/496; A61F 13/505; A61F 13/622; A61F 13/70; A61F 13/78

(57) ABSTRACT

The present disclosure relates to an incontinence apparatus for mitigating the wetting of garments due to involuntary urination in a human male including underpants including a waistband, a pair of leg openings; and a central opening size to allow penetration of a penis; and an absorbent flap positioned on the underpants and being selectively movable between a covering position and a supporting position. The covering position includes the flap being against the underpants and covering the central opening. The supporting position includes the flap having at least one portion secured to the underpants below the central opening with an absorbent flap remaining portion of the absorbent flap extending away from the underpants to be positioned below a penis extending through the opening.

### CROSS REFERENCE TO RELATED APPLICATIONS

This application claims priority to Provisional U.S. Patent Application No. 63/488,404, filed Mar. 3, 2023, the disclosure of which is hereby incorporated by reference in its entirety.

### TECHNICAL FIELD

The present disclosure relates to apparel for the lower body. More particularly, the present disclosure relates to systems and methods for mitigating unwanted wetting in men.

### BACKGROUND

Men and women can develop urinary incontinence for a plurality of reasons that often relate to the muscles and nerves that help the bladder hold or pass urine. Certain examples include weakness in urinary muscles, an inability to communicate signals from the brain to the urinary muscles, blockages or infections in the urinary tract, and trauma to the urinary system. When men experience urinary incontinence, it may lead to unwanted urination at an inappropriate time, which may create an uncomfortable wetting of the man's underpants and/or garments. This may include “dribbling,” which occurs after the conclusion of voluntary urination and after the penis is covered by the man's garments. It is desirable to provide a system for a man with urinary incontinence that can absorb unwanted wetting, comfortably cover the penis, and provide access to the penis when the man is prepared to urinate.

### SUMMARY

The present disclosure relates to apparel for the lower body configured to mitigate unwanted wetting in men. More particularly, the present disclosure relates to a pair of underpants having a central opening size to allow penetration of a penis, an absorbent flap positioned on the underpants and selectively moveable to cover or uncover the central opening, and a pair of trousers configured to be worn on top of the underpants and provide access to the underpants and the absorbent flap through a trouser central opening. The present disclosure is particularly useful for males with muscular disabilities that contribute to urinary incontinence or inhibit a male's ability to urinate in a normal restroom setting. Furthermore, the present disclosure is especially useful for keeping a pair of garments clean from unwanted wetting while preventing fall risks if the floor beneath the male becomes wet and slick.

Furthermore, the present disclosure relates to an incontinence apparatus for mitigating the wetting of garments due to involuntary urination in a human male including underpants including a waistband, a pair of leg openings; and a central opening size to allow penetration of a penis; and an absorbent flap positioned on the underpants and being selectively movable between a covering position and a supporting position. The covering position includes the flap being against the underpants and covering the central opening. The supporting position includes the flap having at least one portion secured to the underpants below the central opening with an absorbent flap remaining portion of the flap extending away from the underpants to be positioned below a penis extending through the opening.

Further yet, the present disclosure relates to a pair of trousers for mitigating the wetting of garments due to involuntary urination in a male including: a trouser waistband; a pair of trouser leg openings; and a trouser central opening size to allow penetration of an absorbent flap, wherein the trouser central opening is selectively movable between a trouser covering position and a trouser supporting position. The trouser covering position includes a trouser flap covering the trouser central opening. The trouser supporting position includes the trouser flap having at least one portion secured to the pair of trousers below the trouser central opening with a trouser remaining portion of the trouser flap extending away from the pair of trousers to be positioned below the absorbent flap extending through the opening.

Further yet, the present disclosure relates to a method of preparing an incontinence apparatus; the method including; providing underpants worn by a male, including a waistband, a pair of leg openings, and central opening sized to accommodate a penis; and removably attaching an absorbent flap to the underpants to selectively cover the central opening, while allowing the absorbent flap to be positioned to project outward from the underpants when the penis extends through the central opening.

Still further yet, the present disclosure relates to an absorbent flap for use with an incontinence apparatus; the absorbent flap including a base material having an elongated shape and a perimeter edge; a fastener material secured to at least a portion of the perimeter edge; and an absorbent core secured to the base material, wherein the flap is shaped and configured to removably cover an opening for a penis in underpants.

A variety of additional aspects will be set forth in the description that follows. The aspects can relate to individual features and to combinations of features. It is to be understood that both the foregoing general description and the following detailed description are exemplary and explanatory only and are not restrictive of the broad inventive concepts upon which the examples disclosed herein are based.

### BRIEF DESCRIPTION OF THE DRAWINGS

The following drawings are illustrative of examples of the present disclosure and therefore do not limit the scope of the present disclosure. Examples of the present disclosure will hereinafter be described in conjunction with the appended drawings, wherein like numerals denote like elements.

FIG. 1 is a front view of a schematic diagram illustrating an example trousers and underpants system, including the pair of trousers, underpants, and absorbent flap being worn on a male.

FIG. 2 is a front view of the underpants of FIG. 1.

FIG. 3 is a front view of the absorbent flap of FIG. 1.

FIG. 4 is a front view of an underpants system in a covering position, including the underpants of FIGS. 1 and 2 and the absorbent flap of FIGS. 1 and 3.

FIG. 5 is a front view of an underpants system in a supporting position, including the underpants of FIGS. 1 and 2 and the absorbent flap of FIGS. 1 and 3.

FIG. 6 is a side view of a schematic diagram of the underpants system of FIGS. 4-5 in the covering position.

FIG. 7 is a side view of a schematic diagram of the underpants system of FIGS. 4-5 in the supporting position.

FIG. 8 is a front view of the trousers of FIG. 1 having a trouser flap covering a trouser central opening.

FIG. 9 is a front view of the trousers of FIG. 1, wherein the trouser flap is not covering the trouser central opening.

FIG. 10 is a side view of a schematic diagram of the trousers of FIGS. 1, 8, and 9 including a trouser flap that is in a covering position.

FIG. 11 is a side view of a schematic diagram of the trousers of FIGS. 1, 8, and 9 including a trouser flap that is in a supporting position.

FIG. 12 is a flow chart illustrating a method for preparing an incontinence apparatus.

### DETAILED DESCRIPTION

Various examples will be described in detail with reference to the drawings, wherein like reference numerals represent like parts and assemblies throughout the several views.

Although the term “male” is sometimes used to refer to a user of the devices disclosed herein, the term is not intended to be so limited in the present disclosure, but rather is intended to broadly include any human that urinates through a penis and/or a catheter.

The present disclosure relates to a system for mitigating unwanted wetting in males, including a pair of trousers, underpants, and an absorbent flap. The terms “pair of trousers” or “trousers” are used herein as a generic term for any type of trousers, pants, slacks, or other clothing having a waistband and two leg openings. The term “underpants” is used herein as a generic term for any type of briefs, boxers, boxer shorts, or other clothing having a waistband and two leg openings that is meant to be worn beneath a pair of trousers.

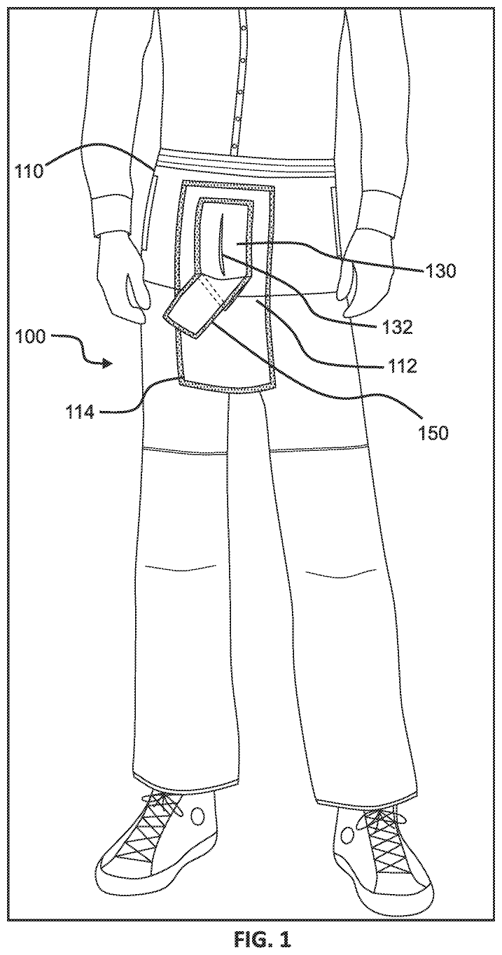

FIG. 1 is a front view of a schematic diagram illustrating an example trousers and underpants system 100, including the pair of trousers (or “trousers”) 110, underpants 130, and absorbent flap 150 being worn on a male.

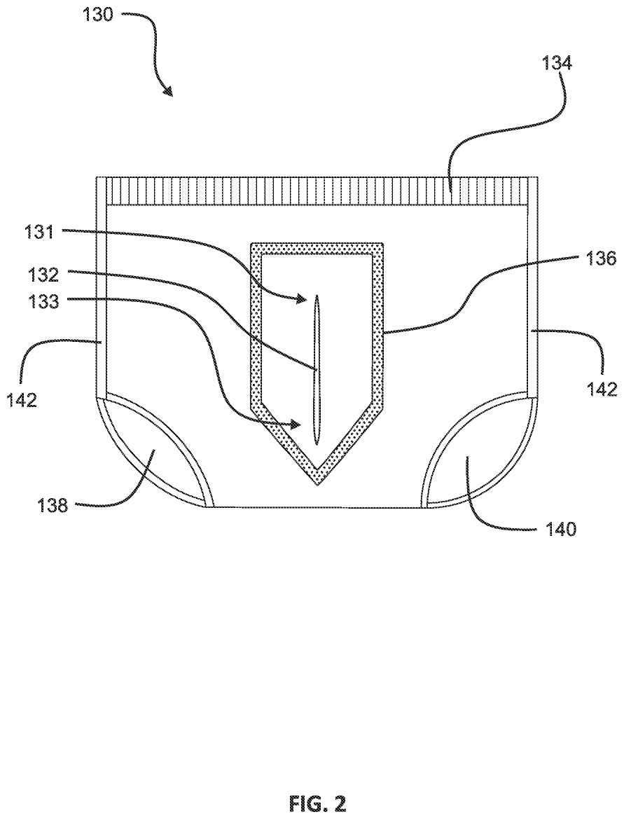

The trousers and underpants system includes underpants 130. The underpants 130 includes an underpants waistband 134, a pair of leg openings 138, 140, and an underpants central opening 132 to allow penetration of a penis. The underpants 130 is configured to be worn beneath the pair of trousers 110 and can be seen in this Figure through the trouser central opening 122. In some embodiments, the underpants 130 is worn in direct contact with the male. The underpants 130 includes an underpants central opening 132 that is configured to allow the male to extend his penis through the opening and urinate without requiring the male to remove the underpants 130 from his waist. This requires less effort than lowering the trousers and underpants system 100 from the male's waist, which is desirable for many men with urinary incontinence that also experience muscular weakness or disorders that affect the nervous system. In some embodiments, the trousers and underpants system 100 includes a coupling arrangement between the absorbent flap and the underpants to permit convenient positioning of the absorbent flap between the covering position and the supporting position. The coupling arrangement includes a fastener 136 positioned between the underpants 130 and the absorbent flap 150 configured to couple the absorbent flap 150 to the underpants 130. In some embodiments, the fastener 136 includes a hook & loop fastener. In some embodiments, the fastener 136 includes at least one button. In some embodiments, the fastener 136 includes an adhesive. In some embodiments, the fastener 136 includes at least one button. The underpants 130 includes a machine-washable fabric that is configured to be washed and sterilized after use. In some embodiments, the machine-washable fabric includes cotton, polyester, nylon, spandex, or some combination thereof. The underpants 130 is illustrated and described in further detail with reference to FIGS. 2 and 4-7.

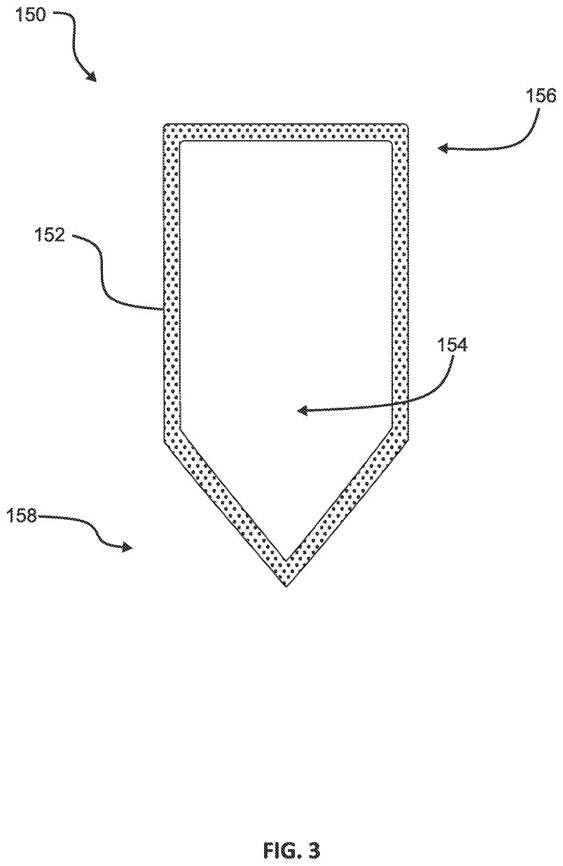

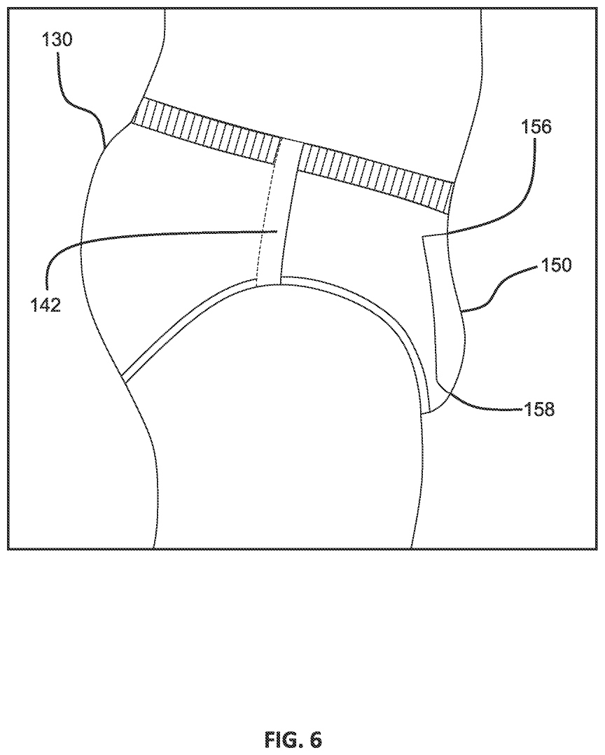

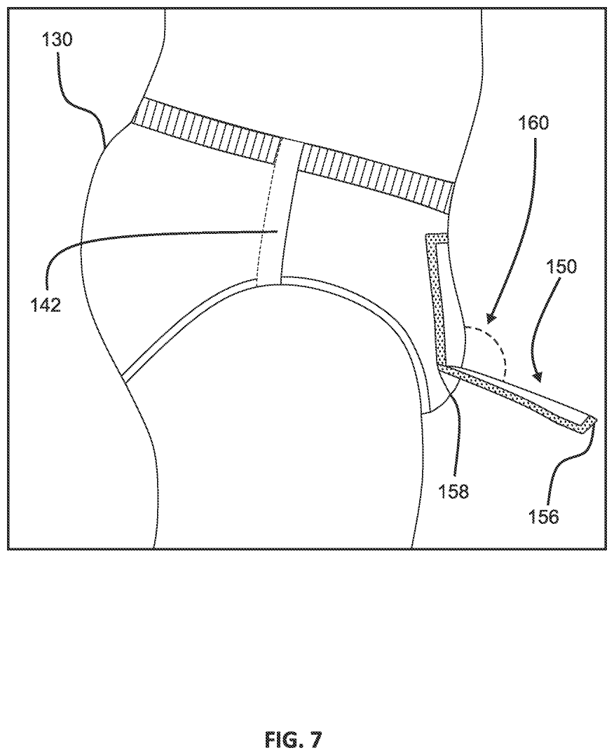

The trousers and underpants system includes an absorbent flap 150. The absorbent flap 150 is positioned on the underpants 130 and is selectively moveable between a covering position and a supporting position. The covering position and the supporting position are illustrated and described herein in further detail with respect to FIGS. 6-7. The absorbent flap 150 is configured to couple to the underpants 130 at the fastener 136. The absorbent flap 150 includes an outer periphery 152, an absorbent core 154, an absorbent flap first end 156, and an absorbent flap second end 158. The outer periphery 152 defines the shape of the absorbent flap 150 while providing support for the penis when the absorbent flap 150 is extended from the underpants 130. In some embodiments, the outer periphery includes an outer periphery fastener that is configured to engage with the fastener 136. In some embodiments, the absorbent core 154 includes felted or woven fibers. In some embodiments, the absorbent core 154 includes a polymer coating such as sodium polyacrylate. In some embodiments, at least one of the absorbent flap first end 156 and the absorbent flap second end 158 are tapered. In some embodiments, the absorbent flap 150 includes a machine-washable fabric that is configured to be washed and sterilized after use. In some embodiments, the machine-washable fabric includes cotton, polyester, nylon, spandex, or some combination thereof. In some embodiments, the absorbent flap 150 is removable and replaceable from the underpants. In some embodiments, the absorbent flap is configured to be disposed of and replaced after use. The absorbent flap 150 is illustrated and described in further detail with reference to FIGS. 3-7.

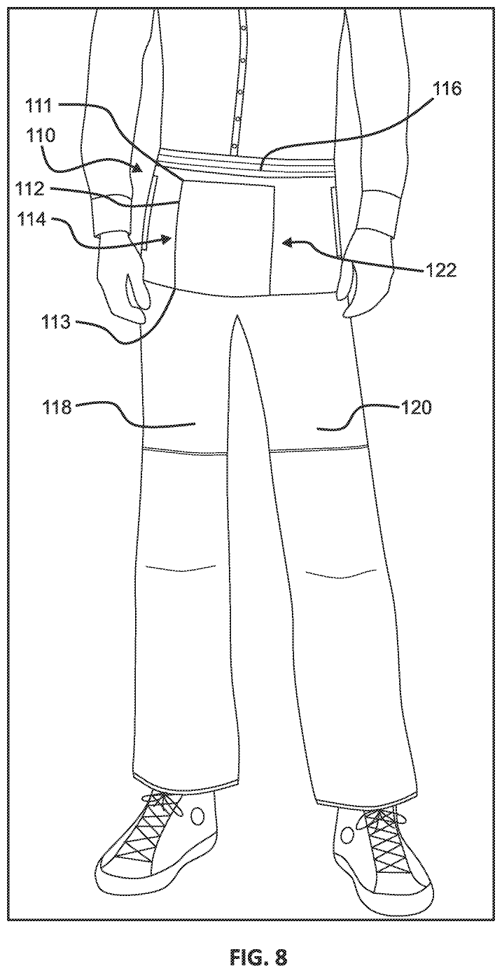

The trousers and underpants system 100 includes a pair of trousers 110 adapted to be worn on top of the underpants 130 and the absorbent flap 150. The pair of trousers includes a trouser waistband 116. The pair of trousers 110 include a pair of trouser leg openings 118, 120. The pair of trousers 110 include a trouser central opening 122 that is selectively covered or exposed by a trouser flap 112. In some embodiments, the trouser flap 112 includes a trouser fastener 114 that is configured to couple the trouser flap 112 to the pair of trousers 110 at a trouser flap first end 111 and a trouser flap second end 113. In some embodiments, the trouser fastener 114 includes a hook & loop fastener. In some embodiments, the trouser fastener 114 includes an adhesive. The trouser flap 112 is configured to attach to, or detach from, the pair of trousers 110 using the trouser fastener 114 to open or close the trouser central opening. When the trouser flap 112 is in an open configuration as shown, an object such as the penis and/or the absorbent flap 150 may penetrate the trouser central opening and allow for the male to urinate. When the trouser flap 112 is in a closed configuration, any object located beneath the trousers is concealed, and objects such as the penis and absorbent flap 150 are not able to penetrate the trouser central opening. The pair of trousers 110 and the alternation between the open and closed configurations are illustrated and described in further detail with reference to FIGS. 8-11.

FIG. 2 is a front view of the underpants 130 of FIG. 1. The underpants 130 includes at least the components as shown, such as the underpants central opening 132, an underpants waistband 134, the fastener 136, a right-leg opening 138, and a left-leg opening 140.

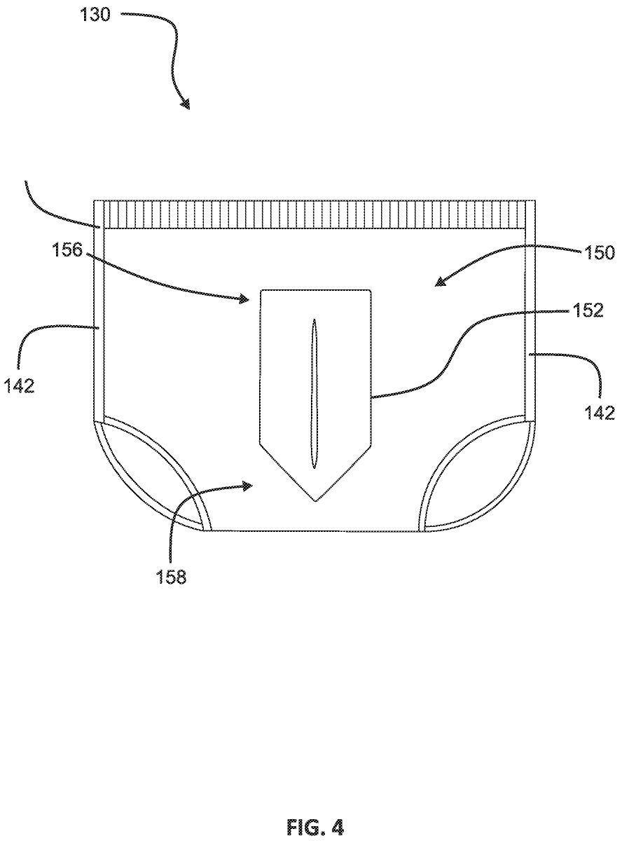

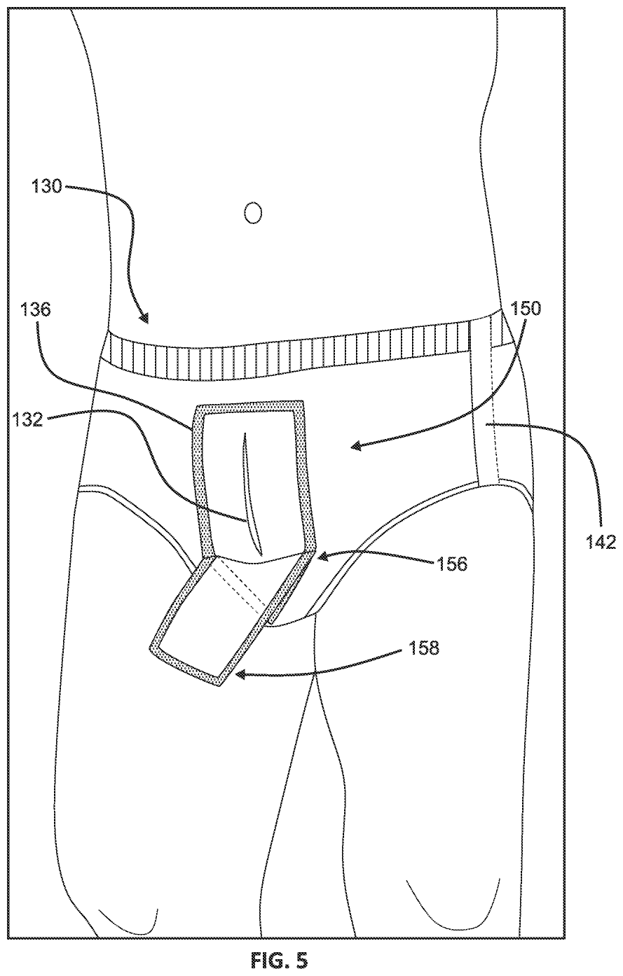

The underpants central opening 132 includes an elongated opening that is substantially centered on a front face of the underpants 130. The underpants central opening 132 is sized such that a distance between an underpants central opening first end 131 and an underpants central opening second end 133 is less than a distance between the absorbent flap first end 156 and the absorbent flap second end 158. This allows for the absorbent flap 150 to completely cover the underpants central opening 132 when the absorbent flap 150 is in a covering position. In some embodiments, the distance between the absorbent flap first end 156 and the absorbent flap second end 158 includes a range between two to five inches. In some embodiments, the distance between the absorbent flap first end 156 and the absorbent flap second end 158 is three-and-a-half inches. The absorbent flap 150 and its positioning on the underpants 130 is illustrated and described in further detail with reference to FIGS. 4-5.

The underpants waistband 134 is positioned on a superior end of the underpants opposite the right-leg opening 138 and the left-leg opening 140. The underpants waistband 134 is configured to secure the underpants to the male about the male's waist. In some embodiments, the underpants waistband 134 includes an elastic material configured to fit around a variety of waist sizes. In some embodiments the elastic waistband includes a braided, knitted, or woven elastic material. In some embodiments, the elastic waistband includes a wrapping material wrapped around at least one rubber core. In some embodiments, the wrapping material includes polyester, cotton, nylon, or a blend of fiber threads.

The fastener 136 is positioned around periphery of the underpants central opening 132 and is configured to couple the absorbent flap 150 to the underpants 130. In some embodiments, the fastener 136 includes a coupling that is positioned around the entire periphery of the underpants central opening 132. In some embodiments, the fastener 136 is positioned around a portion of the underpants central opening 132. In some embodiments, the fastener 136 includes a hook & loop fastener. In some embodiments, the fastener 136 includes an adhesive.

The right-leg opening 138 and the left-leg opening 140 are positioned on opposite ends of the underpants 130 and are configured to allow the male to wear the underpants 130 by penetrating the openings with his legs and positioning the underpants waistband 134 about his waist. In some embodiments, the underpants includes an elastic material that is configured to allow the right-leg opening 138 and the left-leg opening 140 to stretch and accommodate a variety of leg sizes and circumferences.

In some embodiments, the underwear 130 includes side fasteners 142. In some embodiments, the side fasteners 142 include hook & loop fasteners. The side fasteners 142 are configured to couple together to hold the underwear 130 in place around the legs of the male. Furthermore, the side fasteners 142 are configured to decouple to allow the underwear 130 to be easily removed without having to pull both legs through the leg openings 138, 140. This is desirable for patients with limited mobility and provides easy access to the penis.

FIG. 3 is a front view of the absorbent flap 150 of FIG. 1. The absorbent flap 150 includes an outer periphery 152 surrounding an absorbent core 154. Furthermore, the absorbent flap 150 includes an absorbent flap first end 156 for engaging a base of the penis and an absorbent flap second end 158 for engaging a tip of the penis.

The outer periphery 152 is configured to engage with, and couple to, the fastener 136 to couple the absorbent flap to the underpants 130 as shown in FIGS. 4-5. In some embodiments, the outer periphery 152 and the fastener 136 are configured to partially couple such that at least a portion of the outer periphery 152 is coupled to the fastener 136 and at least a portion of the outer periphery 152 is not coupled to the fastener 136. In some embodiments, the outer periphery 152 is configured to completely couple to the fastener 136 to cover the underpants central opening 132. In some embodiments, the outer periphery 152 is configured to completely decouple from the fastener 136 to allow for the absorbent flap 150 to be replaced or cleaned.

The absorbent core 154 is configured to absorb fluid from the penis to mitigate unwanted wetting of the underpants 130 or the trousers 110 in the trousers and underpants system 100. The absorbent core 154 is positioned between the absorbent flap first end 156 and the absorbent flap second end 158. In some embodiments, the absorbent core 154 includes felted or woven fibers. In some embodiments, the absorbent core 154 includes an absorbent polymer coating. In some embodiments, the absorbent polymer coating is sodium polyacrylate. In some embodiments, the absorbent core includes a polyester material.

The absorbent flap first end 156 and the absorbent flap second end 158 engage the underpants 130 to cover the underpants central opening first end 131 and the underpants central opening second end 133 when the absorbent flap is in a covering position. Alternatively, in some embodiments, the absorbent flap 150 is configured to be placed in a supporting position that supports the penis during urination. In the supporting position, the penis penetrates the underpants 130 and is positioned on top of the absorbent flap 150 while the absorbent flap 150 projects from the underpants 130. The covering position is illustrated and described in further detail with reference to FIG. 4. The supporting position is illustrated and described in further detail with reference to FIG. 5. In some embodiments, the absorbent flap is an oblong shape adapted to fully support the penis while in the supporting position. In some embodiments, the oblong shape includes at least one tapered end.

FIG. 4 is a front view of the underpants 130 and absorbent flap 150 in the covering position, including the underpants 130 of FIGS. 1 and 2 and the absorbent flap 150 of FIGS. 1 and 3.

In this Figure, the absorbent flap 150 is at least partially coupled to the underpants 130 at the fastener 136. Specifically, in the covering position, the absorbent flap first end 156 and the absorbent flap second end 158 are coupled to the underpants 130 to cover the underpants central opening 132. The male may wear the underpants 130 and absorbent flap 150 in the covering position to cover the penis when the male does not feel an urge to urinate.

FIG. 5 is a front view of the underpants 130 in the supporting position, including the underpants of FIGS. 1 and 2 and the absorbent flap of FIGS. 1 and 3.

In this Figure, the absorbent flap 150 is partially coupled to the underpants 130 at the fastener 136. Specifically, in the supporting position, the absorbent flap first end 156 is decoupled from the fastener 136 and the absorbent flap second end 158 is coupled to the fastener 136. The absorbent flap first end 156 projects outward to be placed beneath the penis and support the penis during urination. The male uses the underpants 130 and absorbent flap 150 in the supporting position by penetrating the underpants central opening 132 with the penis, decoupling the absorbent flap first end 156 from the fastener 136, resting the penis on top of the absorbent flap 150, and urinating. When the male is finished urinating, the absorbent flap 150 and underpants 130 may be returned to the covering position by returning the penis within the underpants 130 through the underpants central opening 132 and coupling the absorbent flap first end 156 to the fastener.

FIG. 6 is a side view of a schematic diagram of the underpants 130 and absorbent flap 150 of FIGS. 4-5 in the covering position. In this Figure, the absorbent flap 150 is coupled to the underpants 130 at the absorbent flap first end 156 and the absorbent flap second end 158 to cover the underpants central opening 132 and the penis.

FIG. 7 is a side view of a schematic diagram of the underpants 130 and absorbent flap 150 of FIGS. 4-5 in the supporting position. In this Figure, the absorbent flap 150 is coupled to the underpants 130 at the absorbent flap second end 158 with the absorbent flap first end 156 projecting outwardly from the underpants 130 to expose the underpants central opening 132. The absorbent flap first end 156 projects outward at a projection angle 160 formed between the absorbent flap and the underpants 130. In some embodiments, the projection angle 160 includes a range between 0-180 degrees. In some embodiments, the projection angle 160 includes a range between 45-135 degrees. In some embodiments, the underpants 130 includes a support for maintaining the projection angle 160 while the absorbent flap 150 is supporting the penis.

FIG. 8 is a front view of the trousers 110 of FIG. 1 having a trouser flap 112 covering a trouser central opening 122.

In this Figure, the trousers 110 include the trouser flap 112 in a covering position where the trouser fastener 114 couples the trouser flap 112 to the trousers 110 at a trouser flap first end 111 and a trouser flap second end 113. In some embodiments, the trouser fastener 114 includes a hook & loop fastener. In some embodiments, the trouser fastener 114 includes an adhesive coupling. In some embodiments, the trouser fastener 114 outlines at least a portion of a periphery of the trouser central opening 122. In some embodiments, the trouser flap 112 is affixed to the trousers 110 at the trouser flap second end 113.

Furthermore, the trousers 110 include a trouser waistband 116 configured to be worn about the male's waist. In some embodiments, the trouser waistband 116 is adjustable. In some embodiments, the trouser waistband 116 is adjusted by tightening or loosening a drawstring that feeds through the trouser waistband circumferentially about the male's waist.

Furthermore, the trousers 110 include a trouser right leg 118 and a trouser left leg 120. The trouser right leg 118 and the trouser left leg 120 are configured to be worn around the right leg and left leg of the male, respectively. A person of ordinary skill in the art would appreciate the trouser right leg 118, the trouser left leg 120, and the trouser waistband 116 may be created in varying sizes to accommodate males of varying sizes.

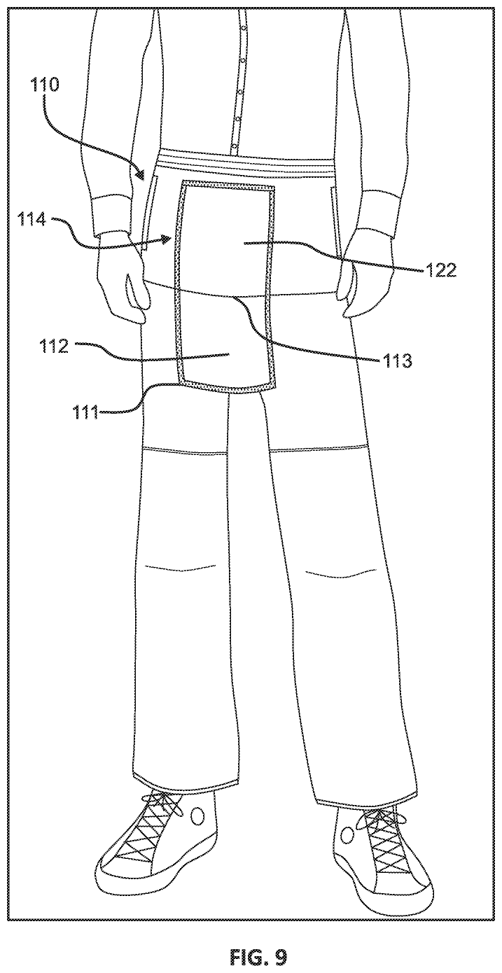

FIG. 9 is a front view of the trousers 110 of FIG. 1, wherein the trouser flap 112 is not covering the trouser central opening 122.

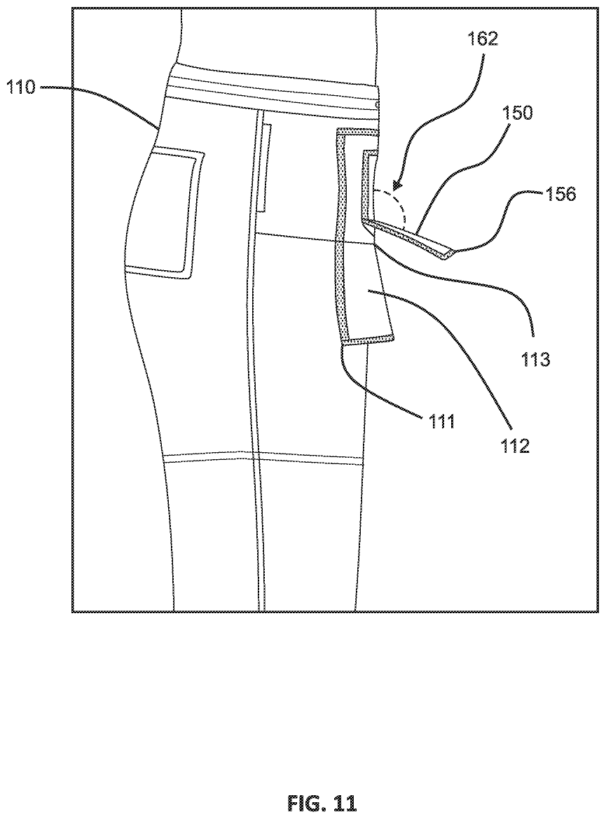

The trouser fastener 114 is visible when the trouser flap 112 is not covering the trouser central opening 122. The trouser central opening 122 can be exposed from beneath the trouser flap 112 by decoupling the trouser flap first end 111 from the trousers 110. When exposed, the trouser central opening 122 is positioned to align with the underpants central opening 132 and the absorbent flap 150. This allows the male to penetrate the underpants central opening 132 and the trouser central opening 122 with the penis while supporting the penis with the absorbent flap 150. An example embodiment of the absorbent flap 150 penetrating the trouser central opening 122 is illustrated and described in further detail with reference to FIG. 11.

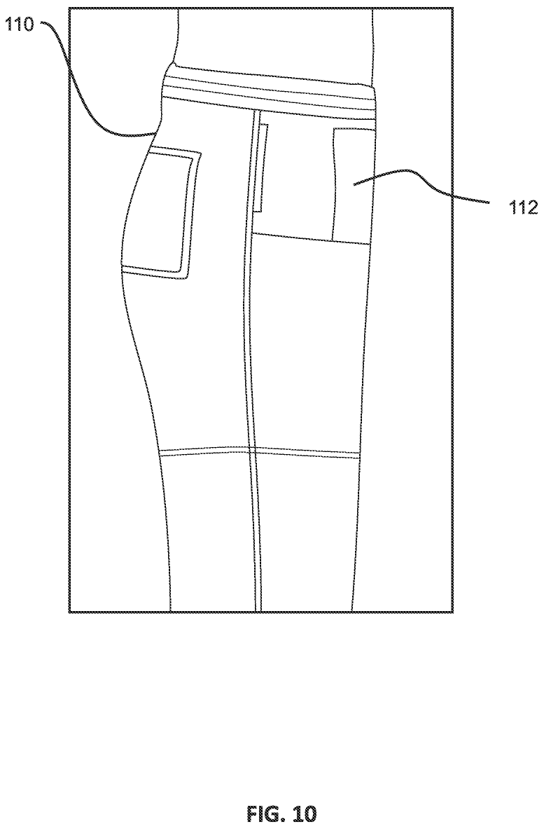

FIG. 10 is a side view of a schematic diagram of the trousers 110 of FIGS. 1, 8, and 9 including a trouser flap 112 that is in a covering position.

In this Figure, the trouser flap 112 is coupled to the trousers 110 at the trouser flap first end 111 and the trouser flap second end 113 to cover the trouser central opening 122. Furthermore, when the trousers are worn within the trousers and underpants system 100, the trouser flap covers the underpants 130, the absorbent flap 150, and the penis while in the covering position.

FIG. 11 is a side view of a schematic diagram of the trousers 110 of FIGS. 1, 8, and 9 including a trouser flap 112 in a supporting position. The trouser flap 112 is decoupled from the trousers 110 at a trouser flap first end 111 to expose the trouser central opening 122. The absorbent flap 150 is coupled to the underpants 130 at the absorbent flap second end 158 with the absorbent flap first end 156 projecting outwardly from the underpants 130 to expose the underpants central opening 132 (not shown). The absorbent flap first end 156 projects outward at a trouser projection angle 162 formed between the absorbent flap 150 and the trousers 110. In some embodiments, the trouser projection angle 162 includes a range between 0-180 degrees. In some embodiments, the trouser projection angle 162 includes a range between 45-135 degrees. In some embodiments, the trousers 110 includes a support for maintaining the trouser projection angle 162 while the absorbent flap 150 is supporting the penis.

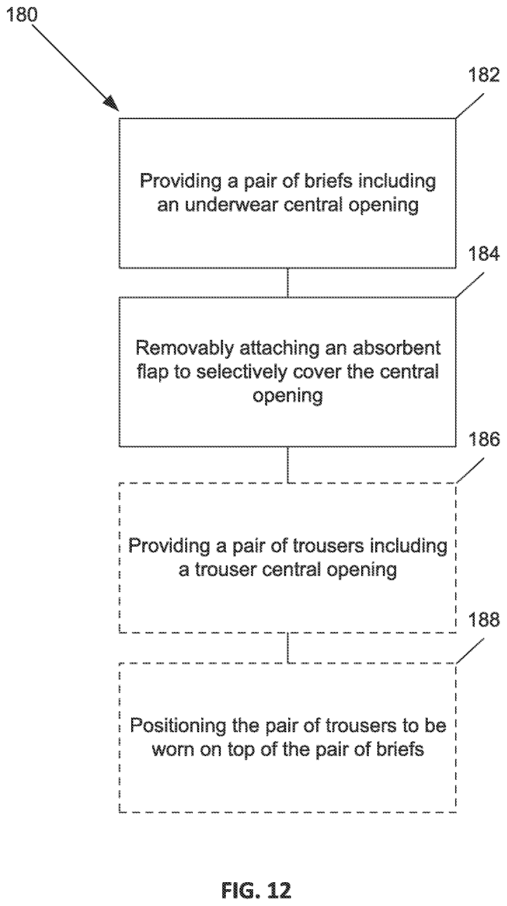

FIG. 12 is a flow chart illustrating a method for preparing an incontinence apparatus 180.

In a first step 182, the underpants 130 is provided that includes an underpants central opening 132. The male may receive the underpants 130 and wear the underpants 130 by inserting the male's right leg through the right-leg opening 138, inserting the male's left leg through the left-leg opening 140, and sliding the underpants waistband 134 around the male's waist.

In a second step 184, the absorbent flap 150 is provided and attached to the underpants 130 to selectively cover the underpants central opening 132. The absorbent flap 150 is coupled to the underpants 130 at the fastener 136. In some embodiments, the absorbent flap is removably coupled to the underpants 130. In some embodiments, the absorbent flap 150 is coupled to the underpants 130 in a covering position. In the covering position, the absorbent flap 150 is coupled to the fastener 136 at the absorbent flap first end 156 and the absorbent flap second end 158 to cover the underpants central opening 132. In some embodiments, the absorbent flap 150 is coupled to the underpants 130 in a supporting position. In the supporting position, the absorbent flap 150 is coupled to the fastener 136 at the absorbent flap first end 156 while the absorbent flap second end 158 is decoupled from the fastener 136. In some embodiments, the absorbent flap 150 is disposable and configured to be disposed of and replaced with a new absorbent flap 150 after use.

In an optional third step, the trousers 110 are provided that include a trouser central opening 122. The male may receive the trousers 110 and wear the trousers 110 by inserting the male's right leg into the trouser right leg 118, inserting the male's left leg into the trouser left leg 120, and sliding the trouser waistband 116 around the male's waist.

In an optional fourth step, the trousers 110 are positioned in the trousers and underpants system 100 by being worn on top of the underpants 130 and the absorbent flap 150 with the underpants central opening 132 and the trouser central opening 122 aligned. In some embodiments, the trouser flap 112 covers the absorbent flap 150 and the underpants 130 in a covering position as illustrated and described in further detail with reference to FIG. 10. In some embodiments, the absorbent flap 150 projects outward from the underpants 130 and penetrates the trouser central opening 122 in a supporting position as illustrated and described in further detail with reference to FIG. 11.

One or more features from any embodiment may be combined with one or more features of any other embodiment without departing from the scope of the invention.

What is claimed is:

1. An incontinence apparatus for mitigating a wetting of garments due to involuntary urination in a human male comprising:
(a) underpants including a waistband, a pair of leg openings; and a central opening to allow penetration of a penis; and
(b) an absorbent flap positioned on the underpants and being selectively movable between a covering position and a supporting position;
(i) the covering position including the absorbent flap being against the underpants and covering the central opening; and
(ii) the supporting position including the absorbent flap having at least one underpants portion secured to the underpants below the central opening with an absorbent flap remaining portion of the absorbent flap extending away from the underpants to be positioned below the penis extending through the central opening.

2. The incontinence apparatus of claim 1, further comprising a coupling arrangement between the absorbent flap and the underpants to permit convenient positioning of the absorbent flap between the covering position and the supporting position.

3. The incontinence apparatus of claim 2, wherein the coupling arrangement includes a hook & loop fastener.

4. The incontinence apparatus of claim 2, wherein the coupling arrangement includes at least one button.

5. The incontinence apparatus of claim 2, wherein the coupling arrangement includes an adhesive.

6. The incontinence apparatus of claim 1, wherein the absorbent flap is removable and replaceable from the underpants.

7. The incontinence apparatus of claim 1, further comprising an absorbent core having felted or woven fibers.

8. The incontinence apparatus of claim 1, wherein the absorbent flap is an oblong shape adapted to fully support the penis while in the supporting position.

9. The incontinence apparatus of claim 1, wherein the absorbent flap is disposable.

10. The incontinence apparatus of claim 1, further comprising a pair of trousers adapted to be worn on top of the underpants, the pair of trousers including:
(a) a trouser waistband;
(b) a pair of trouser leg openings; and
(c) a trouser central opening to allow the penetration of the absorbent flap, wherein the trouser central opening is selectively movable between a trouser covering position and a trouser supporting position;
(i) the trouser covering position including a trouser flap covering the trouser central opening; and
(ii) the trouser supporting position including the trouser flap having at least a portion of the trouser flap secured to the pair of trousers below the central opening with a trouser remaining portion of the trouser flap extending away from the pair of trousers to be positioned below the absorbent flap extending through the trouser central opening.

11. The incontinence apparatus of claim 10, wherein the trouser flap is at least partially secured to the pair of trousers with double stitching.

12. A pair of trousers for mitigating a wetting of garments due to involuntary urination in a human comprising:
(a) a trouser waistband;
(b) a pair of trouser leg openings; and
(c) a trouser central opening to allow penetration of an absorbent flap, wherein the trouser central opening is selectively movable between a trouser covering position and a trouser supporting position;
(i) the trouser covering position including a trouser flap covering the trouser central opening; and
(ii) the trouser supporting position including the trouser flap having at least one portion of the trouser flap secured to the pair of trousers below the trouser central opening with a trouser remaining portion of the trouser flap extending away from the pair of trousers to be positioned below the absorbent flap extending through the trouser central opening.

13. The pair of trousers of claim 12, wherein a trouser covering is disposed around a periphery of the trouser central opening with double stitching.

14. The pair of trousers of claim 12, wherein the trouser waistband is adjustable.

15. The pair of trousers of claim 12, further comprising underpants adapted to be worn underneath the pair of trousers, the underpants including:
(a) a waistband, a pair of leg openings; and an underpants central opening to allow the penetration of a penis; and
(b) the absorbent flap positioned on the underpants and being selectively movable between a covering position and a supporting position;
(i) the covering position including the absorbent flap being against the underpants and covering the underpants central opening; and
(ii) the supporting position including the absorbent flap having at least one underpants portion secured to the underpants below the underpants central opening with an absorbent flap remaining portion of the absorbent flap extending away from the underpants to be positioned below the penis extending through the underpants central opening.

16. The pair of trousers of claim 15, wherein the pair of trousers are machine washable.

17. A method of preparing an incontinence apparatus comprising:
(a) providing underpants worn by a human, including a waistband, a pair of leg openings, and central opening sized to accommodate a penis; and
(b) removably attaching an absorbent flap to the underpants to selectively cover the central opening, while allowing the absorbent flap to be positioned to project outward from the underpants when the penis extends through the central opening.

18. The method of claim 17, further comprising providing a pair of trousers worn by the human on top of the underpants, the pair of trousers including: a trouser waistband; a pair of trouser leg openings; and a trouser central opening sized to accommodate the central opening.

19. The method of claim 18, further comprising allowing the absorbent flap to be positioned to project through the trouser central opening when the penis extends through the trouser central opening.

20. An absorbent flap for use with an incontinence apparatus; the absorbent flap comprising:
(a) a base material having an elongated shape and a perimeter edge;
(b) a hook & loop fastener material secured to at least a portion of the perimeter edge; and
(c) an absorbent core secured to the base material; wherein the absorbent flap is shaped and configured to removably cover an opening for a penis in underpants.
